(19) United States
(12) Patent Application Publication
(10) Pub. No.: US 2023/0277462 A1
(43) Pub. Date: Sep. 7, 2023

(54) WHOLE CANNABIS PRODUCT COMPOSITIONS AND METHODS OF MANUFACTURE WITHOUT SPECIFIC DECARBOXYLATION OF THE WHOLE PLANT CONSTITUENTS

(71) Applicant: Thomas Vella, Scottsdale, AZ (US)

(72) Inventor: Thomas Vella, Scottsdale, AZ (US)

(21) Appl. No.: 18/097,222

(22) Filed: Jan. 14, 2023

Related U.S. Application Data
(63) Continuation-in-part of application No. 17/352,278, filed on Jun. 19, 2021, now abandoned.
(60) Provisional application No. 63/266,833, filed on Jan. 14, 2022.
(60) Provisional application No. 63/041,790, filed on Jun. 19, 2020.

(51) Int. Cl. A61K 9/16; A61K 36/185

(52) U.S. Cl. CPC A61K 9/1688; A61K 36/185

(57) ABSTRACT

A cannabis powder composition and method of manufacture which is a non-extracted botanical ingredient that is manufactured to preserve the natural levels of beneficial elements found in the cannabis plant including cannabinoids, terpenes, flavonoids, essential vitamins, fatty acids, protein and fiber. The cannabis powder is produced using a low heat technology which controls decarboxylation and allows the product to remain in an active and highly bioavailable state

### BACKGROUND OF THE INVENTION

### Field of the Invention

The present invention relates to a cannabis powder composition and method of manufacture. More particularly, the invention relates to a cannabis powder composition containing a variety of beneficial constituents and a method of manufacture that does not chemically modify, and specifically controls decarboxylation without chemicals, the beneficial constituents such as for example, cannabinoid rates.

### Description of the Related Art

Cannabis products particularly, THC products, have become increasingly common due to however increase in THC overdoses are becoming prevalent in states that have legalized and made cannabis products recreational. Study is ongoing regarding the benefits of cannabis products. It has more recently been determined that other components of the cannabis plant, not just the THC, may have beneficial effects. It is also believed that the efficacy of various cannabinoids, and other compounds found in cannabis, may depend on the extent to which they are altered or modified during the extraction process. Some extraction process may substantially reduce the efficacy of the final product due to modifications caused by extraction methods that include high temperatures and/or solvents. Methylation, decarboxylation, denaturation, reduction and oxidation are just a few types of chemical modifications that the components of the cannabis plant may undergo during extraction from a plant to produce a concentrated extract, oil or powder.

The above-described deficiencies of today's systems are merely intended to provide an overview of some of the problems of conventional systems, and are not intended to be exhaustive. Other problems with the state of the art and corresponding benefits of some of the various non-limiting embodiments may become further apparent upon review of the following detailed description.

In view of the foregoing, it is desirable to provide a method of synthesizing a powdered form of cannabis that keeps in tact the entire plant material in tact along with the naturally occurring cannabinoids. More particularly, it is desirable to provide a cannabis powder product that is formed using methods that control decarboxylation of the components of the cannabis plant.

### BRIEF SUMMARY OF THE INVENTION

Disclosed is a cannabis powder which is a non-extracted botanical ingredient that is manufactured to preserve the natural levels of beneficial elements found in the cannabis plant including cannabinoids, terpenes, flavonoids, essential vitamins, fatty acids, protein and fiber. The cannabis powder is produced using a low heat technology which controls decarboxylation and allows the product to remain in an active and highly bioavailable state delivering the “Entourage Effect” in its truest form.

Beyond the 113 known phytocannabinoids, the cannabis plant also contains terpenes, flavonoids, essential vitamins, fatty acids, protein and fiber. When consumed together as a whole, a synergistic effect takes place producing increased efficacy and a better more controlled THC high. This is known as the “Entourage Effect”. The present invention provides a cannabis powder and method of preparing the cannabis powder that controls unwanted chemical modification of the various beneficial constituents of cannabis. In particular, the processes disclosed herein control decarboxylation of the cannabinoids as well as other components. Unlike other products and methods of production, the process disclosed herein maintains the integrity of the cannabis plant in order to create an efficacious and all-natural THC product that can be dispersed more evenly than THC oils for dosing purposes. This Whole THC Powder may be utilized to formulate in manufacture capsules, tablets, tinctures, powders, gummies, cookies, bars, coffee, teas, drinks, candies, products and the like.

It is therefore an object of the present invention to provide a cannabis powder produced from all-natural sources containing no pesticides, synthetic components or chemical additives and is safe for human consumption. The product is not treated with ethylene oxide and is unadulterated.

These and other objects and advantages of the present invention will become apparent from a reading of the attached specification and appended claims. There has thus been outlined, rather broadly, the more important features of the invention in order that the detailed description thereof that follows may be better understood, and in order that the present contribution to the art may be better appreciated. There are features of the invention that will be described hereinafter and which will form the subject matter of the claims appended hereto.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is a FIG. 1 is graph showing the efficacy of a whole cannabis plant product compared to the efficacy of a THC isolate or oil in accordance with the principles of the invention;

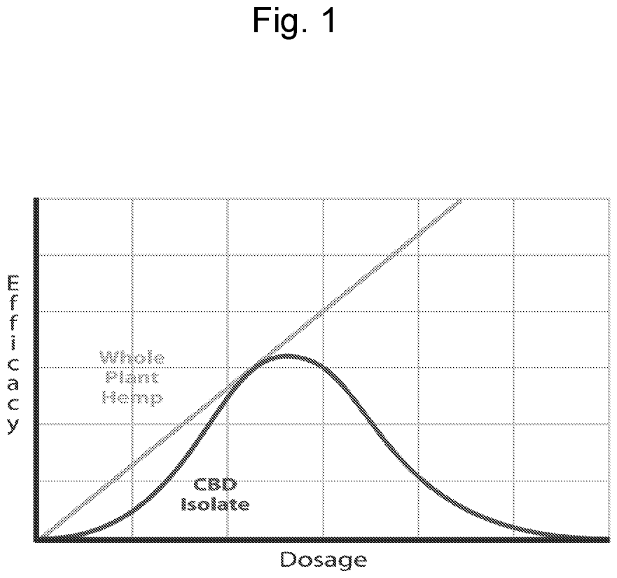

FIG. 2 is a chart summarizing the qualities of a whole cannabis powder product manufactured in accordance with the principles of the invention;

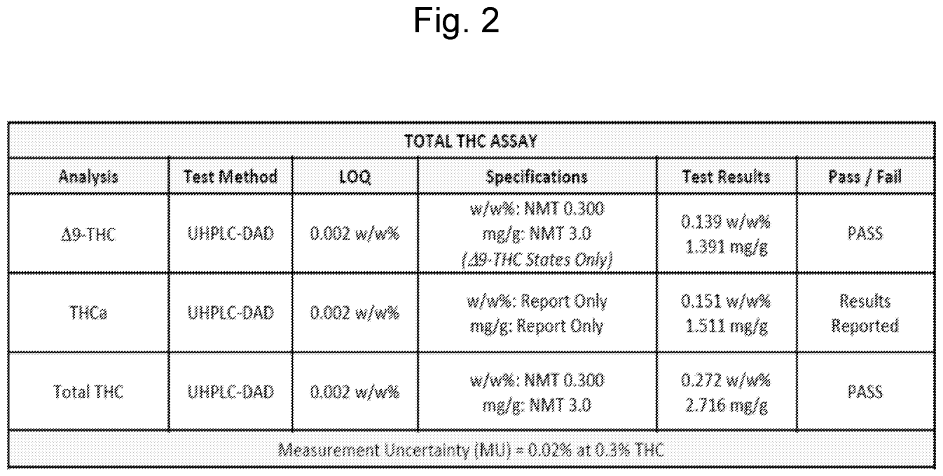

FIG. 3 is a chart showing the results of a cannabinoid assay of a whole cannabis product manufactured in accordance with the principles of the invention;

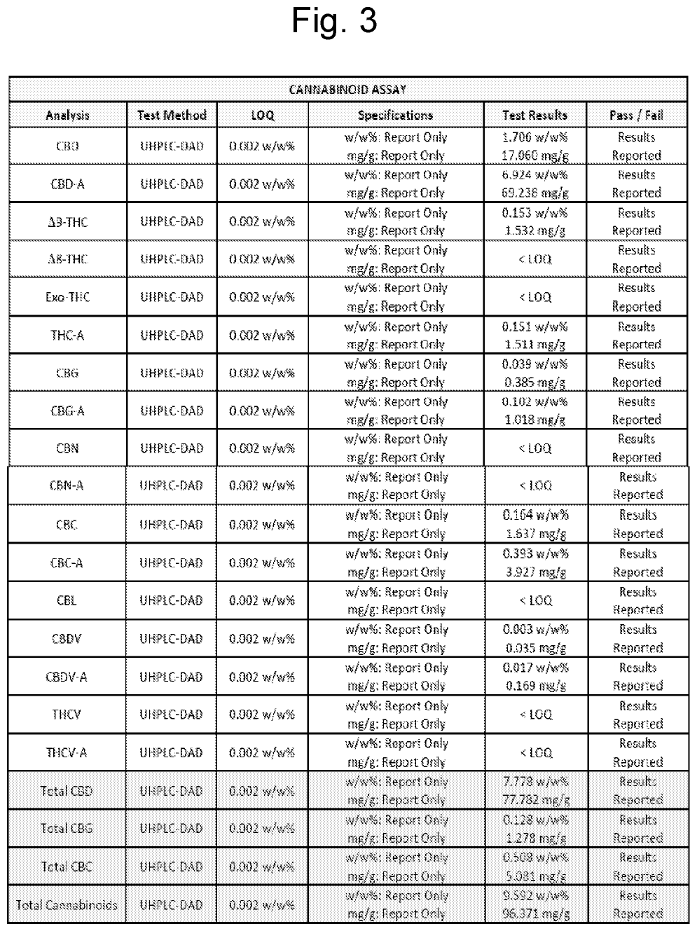

FIG. 4 is a chart showing the results of a heavy metal assay of a whole cannabis product manufactured in accordance with principles of the invention;

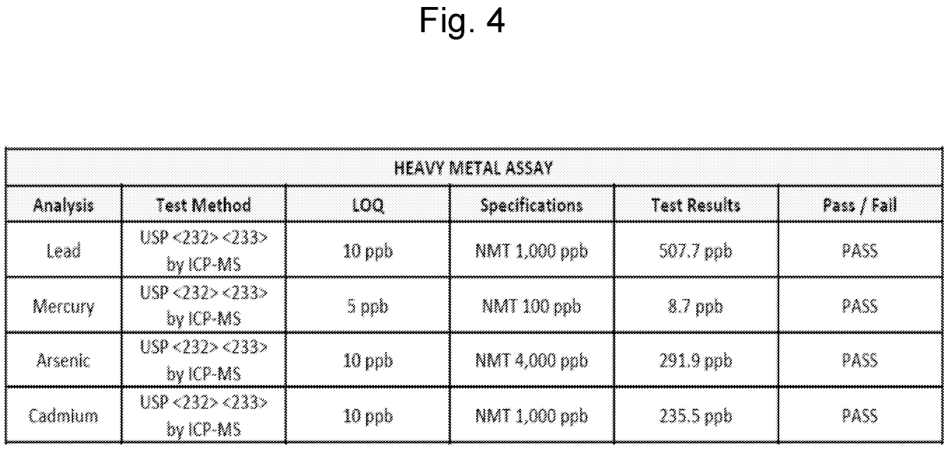

FIG. 5 is a chart showing the results of microbiological and mycotoxin assays of a whole cannabis product manufactured in accordance with principles of the invention;

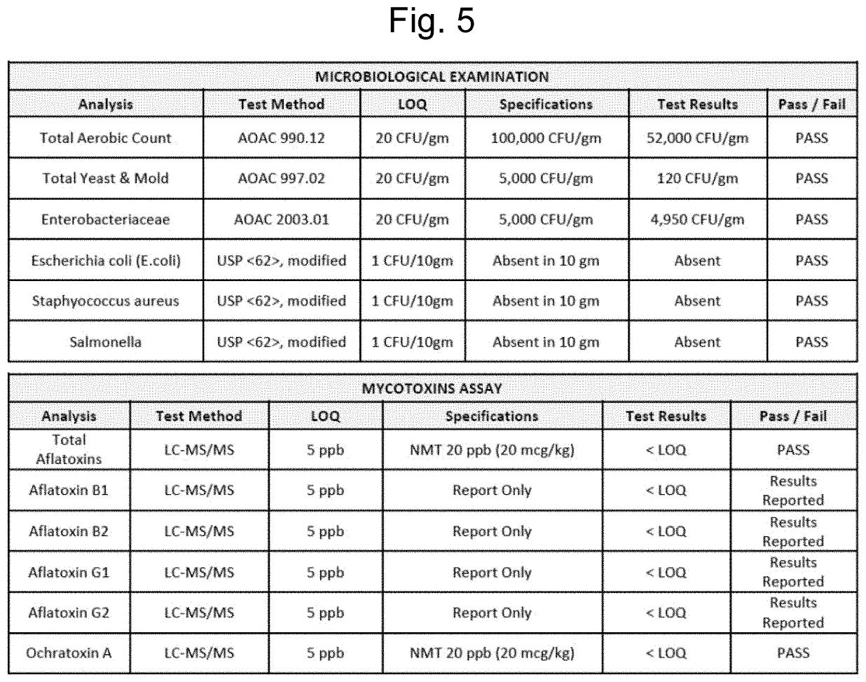

FIG. 6 is a chart summarizing the results of testing of the finished whole cannabis product manufactured in accordance with the principles of the invention;

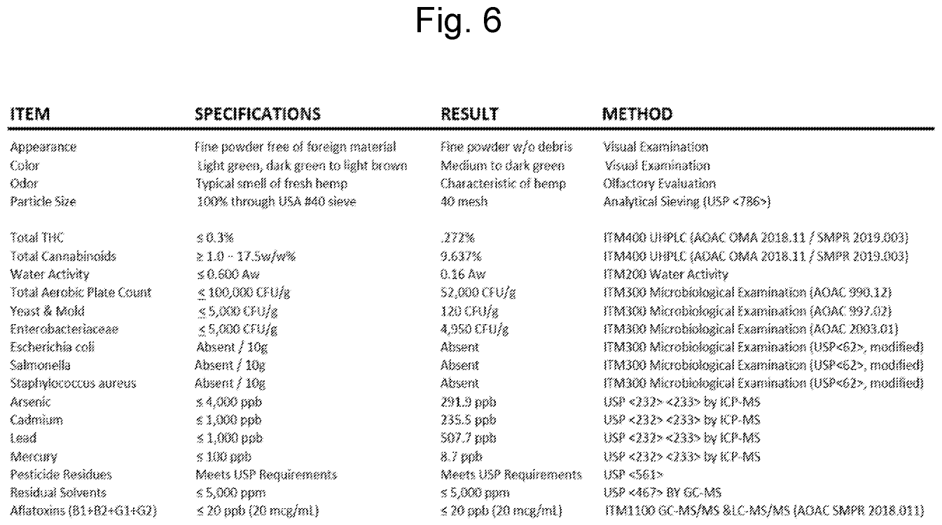

FIG. 7 is another chart summarizing further results of testing of the finished whole cannabis product manufactured in accordance with principles of the invention.

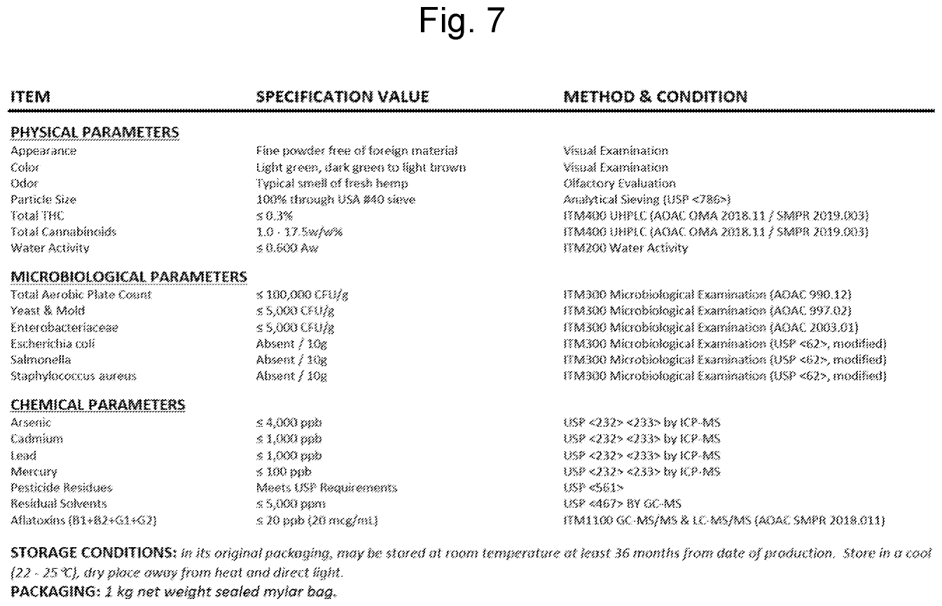

### DETAILED DESCRIPTION

The invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

The disclosed subject matter is described with reference to the drawings, wherein like reference numerals are used to refer to like elements throughout. In the following description, for purposes of explanation, numerous specific details are set forth in order to provide a thorough understanding of the various embodiments of the subject disclosure. It may be evident, however, that the disclosed subject matter may be practiced without these specific details. In other instances, well-known structures and devices are shown in block diagram form in order to facilitate describing the various embodiments herein. Various embodiments of the disclosure could also include permutations of the various elements recited in the claims as if each dependent claim was a multiple dependent claim incorporating the limitations of each of the preceding dependent claims as well as the independent claims. Such permutations are expressly within the scope of this disclosure.

Unless otherwise indicated, all numbers expressing quantities of ingredients, dimensions, reaction conditions and so forth used in the specification and claims are to be understood as being modified in all instances by the term “about”. The term “a” or “an” as used herein means “at least one” unless specified otherwise. In this specification and the claims, the use of the singular includes the plural unless specifically stated otherwise. In addition, use of “or” means “and/or” unless stated otherwise. Moreover, the use of the term “including”, as well as other forms, such as “includes” and “included”, is not limiting. Also, terms such as “element” or “component” encompass both elements and components comprising one unit and elements and components that comprise more than one unit unless specifically stated otherwise.

Various embodiments of the disclosure could also include permutations of the various elements recited in the claims as if each dependent claim was a multiple dependent claim incorporating the limitations of each of the preceding dependent claims as well as the independent claims. That is, the combinations of the various components of the invention are not limited to those combinations expressly shown in the Figures. Unless expressly stated otherwise, components described in one embodiment may be interchanged with components of the same name found in other embodiments. Such permutations are expressly within the scope of this disclosure.

The cannabis powder is formed from Cannabis sativa or similar plants, using the buds, flowers and leaves of the plants. No solvents are used, as it not an extraction method. The final product is a 40-80-mesh powder, with a light green color and fresh odor. It ranges between 18% to 33% THC, with a total cannabinoid content of 1.0-17.5%.

The cannabis powder product is manufactured using a method that preserves the natural levels of beneficial elements found in the cannabis plant including cannabinoids, terpenes, flavonoids, essential vitamins, fatty acids, protein and fiber. It generally should be administered in average doses of approximately in 10 mg of total THC content.

The cannabis powder product manufactured by the processes disclosed herein includes important and beneficial constituents of the cannabis plant that work synergistically to increase efficacy due to the “entourage effect” by providing substantially all of the beneficial ingredients without chemical or other substantial modifications.

Process—Cannabis is grown until fully matured. The cannabis is weighed and placed in quarantine until testing protocols are completed. The potency for THC is tested along with the full panel of cannabinoids. Once testing is completed the material is released for processing it is issued an internal lot number.
Step 1—The cannabis biomass/buds goes through a metal detector and magnet to remove impurities.
Step 2—The cannabis is placed in a containment unit that holds up to 3000 lbs. The cannabis is carefully cleaned with an organic solvent-free solution and dried at a temperature of 245 degrees Fahrenheit or less for a specific amount of time.
Step 3—The cannabis biomass is pulverized into a fine powder.
Step 4—The cannabis biomass is spun and dried with low humidity air (very dry air) at 245 degrees Fahrenheit or less for a specific amount of time.

This step is important to preserving all of the cannabinoids that are naturally occurring in the cannabis plant and controlling the decarboxylation. The reason why the temperature and dry air for the allocated time are so important is to control the, THCa, and the other cannabinoids in their original acidic state. CBDa and CBGa are preserved through this process as a whole food powder, unlike the other processes that use extreme heat and chemicals to change the molecular structure of the plant typically only focusing on the THC content. This process keeps the cannabis plant that is rich in cannabinoids intact and this is why Whole Plant THC delivers a better smoother high than extracted THC. This process is the way Mother Nature intended THC to be consumed.
Step 5—The fine Raw Cannabis Powder is passed through a metal detector and magnet to remove impurities.
Step 6—The fine powder is assigned a final finished goods lot number and samples of the powder are sent out to a DEA-certified testing facility for potency, microbial counts, heavy metals as well as other tests to confirm it is within our finished goods specifications.
Step 7—The fine powder is packaged in airtight food-grade containers and ready to be sold commercially.

In another optional process in accordance with principles of the invention, the premium parts of the plant are used—primarily buds and some leaves but no stems or stalks.
Step 1—the raw product is analyzed, where it is tested for THC, yeast mold, coliform, Total Plate Count etc. Only raw product was suitable qualities is used. If the biomass passes all tests is used in the following steps.
Step 2—Cannabis is put in massive blender where it is sprayed with a very fine mist of food grade alcohol.
Step 3—Cannabis is dried at low heat −240 degrees or less.
Step 4—Cannabis is pulverized into a fine powder.
Step 5—Cannabis is sifted to desired mesh size.
Step 6—Cannabis is packaged in waterproof containers.

The product produced by the above processes is all natural, contains no pesticides, synthetic components or chemical additives and is safe for human consumption. It is not treated with ethylene oxide (EtO) and is certified to be free of any defects, solvents and foreign materials. It is useful for production of products such as food and beverages including bars, coffee, tea, carbonated soft drinks, fruit drinks, nutrition powders, energy drinks, candies and ice cream, with generally a maximum addition levels ranging from 0.05-1.5 mg/g, depending upon the specific category. It is suitable for use as a dietary ingredients in dietary supplements, and has less than 0.3% THC.

The cannabis product manufactured using the above-described processes is not an isolate. It is a non-extracted powder created using technology designed to maintain the functional elements within botanicals at their natural levels. It is generally made from the buds, flowers and leaves of industrial cannabis that may be grown in the USA and is free of heavy metals, residual solvents and pesticides. The cannabis product is <0.3% Total THC making it federally legal under the 2018 Farm Bill. It is also Non-GMO, Gluten Free and Vegan.

Whereas, the present invention has been described in relation to the drawings attached hereto, other and further modifications, apart from those shown or suggested herein, may be made within the spirit and scope of this invention. Those skilled in the art will appreciate that the conception, upon which this disclosure is based, may readily be utilized as a basis for the designing of other structures, methods and systems for carrying out the several purposes of the present invention. Descriptions of the embodiments shown in the drawings should not be construed as limiting or defining the ordinary and plain meanings of the terms of the claims unless such is explicitly indicated. The claims should be regarded as including such equivalent constructions insofar as they do not depart from the spirit and scope of the present invention.

1. A method of processing whole cannabis plants comprising:
a) sending whole cannabis plants through a metal detector and magnet;
b) cleaning the whole cannabis with an organic solvent-free solution and dried at a temperature of 245 degrees Fahrenheit or less for a specific amount of time;
c) pulverizing the whole cannabis into a fine whole cannabis powder;
d) drying the whole cannabis powder by spinning in low humidity air at 245 degrees Fahrenheit or less for a specific amount of time;
e) sending the whole cannabis powder through a metal detector and magnet; and,
f) packaging the whole cannabis powder in airtight food-grade containers and ready to be sold commercially.
